SEQUENCE LISTING (19 sequences)

SEQ ID NO: 1 - protein, 144 aa, Oryctolagus cuniculus
MWLQNLFLLGSVVCTISAPTHQPNTVSQPLKHVDAIKEARIILSRSNDSAAVPGEMVEVVSEMFDPQKPTCLQTRLELYKQGLRGSLERLSSTLTLMASHYKQNCPPTPETSCETEFITFKSFKENLKCFLFVIPFNCWEPVQK

SEQ ID NO: 2 - DNA, 657 bp, Oryctolagus cuniculus
aaggctaaggtcctgaggaggatgtggttgcagaacctgttcctcctaggcagtgtggtctgcaccatctctgcacccacccaccagcccaacactgtcagccagcccttgaagcatgtggatgccatcaaggaggcccggatcatcctgagccgcagtaacgattctgccgctgtgccgggcgaaatggtagaagtcgtctctgaaatgtttgatcctcagaaaccaacttgcctgcagacccgcctggaactgtacaagcaaggcctgcggggcagcctggagcggctctcgagtaccctgactttgatggccagccactacaagcaaaactgtcccccaaccccggaaacttcctgtgagaccgagtttatcaccttcaaaagtttcaaagagaacctgaagtgctttctgtttgtcatcccctttaactgctgggagccagtccagaagtgaggaagcgcaggctagccaggccagccctggttgttgacctcagagactactgctctcccacccaaaagagccaaaaactcaggatcttcgtgttggagggaccaaggggccactgtggggacagcatggacctgccctggaccacactgacccagttatggacctgccctggaccacactgacccagttatggacctgccc

SEQ ID NO: 3 - DNA, 26 bp, Artificial Sequence
  Primer
aagcagtggtatcaacgcagagtact

SEQ ID NO: 4 - DNA, 21 bp, Artificial Sequence
  Primer
aaggctaaggtcctgaggagg

SEQ ID NO: 5 - DNA, 45 bp, Artificial Sequence
  Primer
ctaatacgactcactatagggcaagcagtggtatcaacgcagagt

SEQ ID NO: 6 - DNA, 22 bp, Artificial Sequence
  Primer
ctaatacgactcactatagggc

SEQ ID NO: 7 - protein, 144 aa, Homo sapien
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 8 - protein, 141 aa, Mus musculus
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 9 - protein, 144 aa, Felis
MWLQNLLFLNTVVCSISAPTSSPSSVTRPWQHVDAMKEALSLLNNSSEITAVMNETVEVVSEMFDPEEPKCLQTHLKLYEQGLRGSLISLKEPLRMMANHYKQHCPLTPETPCETQTITFKNFKEKLKDFLFNNPFDCWGPDQK

SEQ ID NO: 10 - protein, 146 aa, Equus
MWLQNLLLLGTVVYSMPAPTRQPSPVTRPWQHVDAIKEALSLLNNSSDTAAIMNETVEVVSETFDAEELTCLQTRLKLYKQGLRGSLIKLEGPLTMMASHYKQHCPPTLETSCATQMITFKSFKKNLKDFLFEIPFDCWKPAQKLE

SEQ ID NO: 11 - protein, 144 aa, Macaca
MWLQGLLLLGTVACSISAPARSPSPGTQPWEHVNAIQEARRLLNLSRDTAAEMNKTVEVVSEMFDLQEPSCLQTRLELYKQGLQGSLTKLKGPLTMMASHYKQHCPPTPETSCATQIITFQSFKENLKDFLLVIPFDCWEPVQE

SEQ ID NO: 12 - protein, 144 aa, Sus
MWLQNLLLLGTVVCSISAPTRPPSPVTRPWQHVDAIKEALSLLNNSNDTAAVMNETVDVVCEMFDPQEPTCVQTRLNLYKQGLRGSLTRLKSPLTLLAKHYEQHCPLTEETSCETQSITFKSFKDSLNKFLFTIPFDCWGPVKK

SEQ ID NO: 13 - protein, 144 aa, Papio
MWLQGLLLLGTVACSISAPARLPSPGMQPWEHVNAIQEARRLLNLSRDTAAEMNKTVEVVSEMFDLQEPSCVQTRLELYKQGLQGSLTKLKGPLTMMASHYKQHCPPTPETSCATQIITFQSFKEDLKDFLLVIPFDCWEPVQE

SEQ ID NO: 14 - protein, 143 aa, Bubalus
MWLQNLLLLGTVVCSFSAPTRPPSTVTRPWQHVDAIKEALSLLNQSSEPDAGMNDTEVVSEMFDAQEPTCLQTRLKLYKKGLQGSLTSLMGSLTMMATHYEKHCPPTPETSCGTQFITFKSFKEDLKEFLFIIPFDCWEPAQK

SEQ ID NO: 15 - protein, 144 aa, Cervus
MWLQNLLLLGTVVCSFSAPTRPASPVTRPWQHVDAIKEALSLLNHSSDTAAVMNETVEVVSEPFDSQEPTCLQTRLKLYKQGLRGSLTSLSGSLTMMARHYEQHCPPTQETSCETQTITFKSFKENLKDFLFIIPFDCWEPAQK

SEQ ID NO: 16 - protein, 145 aa, Meriones
MWLQNLLFLSIVVYSFSAPTHSPITVTQPWKHVDAIKEALSLLEKMLKIPAMLDEDDVDIVSEEFSVQRPTCLQKRLKVYEQGLRGNFTRFRGTLAMIARHYQKYCPPTPEDECETEVTTFGDFIDSLKNFLFDIPFDCWEPVQE

SEQ ID NO: 17 - protein, 145 aa, Cavia
MWLQNLLLLGTVVCSICAPTDLLSPVTQSWKHVDATINEALSLLNHTSDPAAVMNETVEVVYDQFEPQEPTCLQTRLALFMKGLRGNLTRLEGSLTLMANFYKQHCPPTPETSCMTQIITFKSFKENLKRFLFAIPFDCWEPVQK

SEQ ID NO: 18 - protein, 141 aa, Sigmodon
MWLQFLLFLGIVVCSFSAPTRSPASVTRPWKHVDAIKEALSLLNDMPAMENEDVDIVSKEFSIQRPTCVQTRLKVYQQGLQGNFTKLKGALNMMASHYQKNCPPTPEIDCETQVTTFEDFIDNLKGFLFDIPFDCWKPVQK

SEQ ID NO: 19 - protein, 90 aa, Peromyscus
MWLKILLFLGIVVCSFSAPTRSPAPVTQPWNHVEAIKEALILLDNAPDIVSEDEDVEIVSEEFSVQKCVQERLQLYEKGLRGNLTKLKGA

(19) United States
(12) Patent Application Publication
(10) Pub. No.: US 2006/0153800 A1
(43) Pub. Date: Jul. 13, 2006

(54) DNA immunization with recombinase/transposase

(76) Inventors: Roland Buelow, Palo Alto, CA (US); Josef Platzer, Geretsried (DE)

Correspondence Address: HELLER EHRMAN LLP, 275 MIDDLEFIELD ROAD, MENLO PARK, CA, 94025-3506, US

(21) Appl. No.: 11/301,800

(22) Filed: Dec. 12, 2005

Related U.S. Application Data
(60) Provisional application No. 60/636,361, filed on Dec. 14, 2004.

(51) Int. Cl. C07K 14/535; A61K 48/00; A61K 38/19; C07K 16/24; C07H 21/04; C12P 21/02

(52) U.S. Cl. 424/85.1; 435/69.5; 435/320.1; 435/325; 530/351; 530/388.23; 514/44; 536/23.5

(57) ABSTRACT

The present invention relates to improved methods to immunize/vaccinate or stimulate the immune system of animals, including humans, using vectors containing expression cassettes that encode for the DNA of one or more protein/peptide antigens and/or adjuvants, in particular, cytokines like GMCSF, Flt3L, interleukins, and the like, which can be encoded by DNA as well, also recombinase mediated integration. Adjuvants known to increase immune responses following DNA vaccination. In addition, the vectors contain one or more sites recognized by a recombinase/transposase, which catalyzes the insertion of the vector into the genome of transfected cells. Stable integration of the plasmid vector into the genome of transfected cells results in higher and longer-lasting expression of the encoded protein(s), and increases the immune response in the vaccinated animal. The present invention also relates to adjuvant compositions comprising the novel polypeptide, rabbit GMCSF, for boosting antibody production in rabbits.

### CROSS-REFERENCE TO RELATED APPLICATIONS

This is a non-provisional application filed under 37 C.F.R. §1.53(b), claiming priority under U.S.C. Section 119(e) to U.S. Provisional Patent Application Ser. No. 60/636,361 filed Dec. 14, 2004.

### FIELD OF THE INVENTION

This invention relates to the field of DNA vaccines and adjuvants. Specifically, the present invention relates to improved methods to immunize/vaccinate or stimulate the immune system of animals, including humans, using vectors containing expression cassettes that encode one or more protein/peptide antigens and/or adjuvants and a recombinase that mediates the integration of the DNA into the genome of the animal. The present invention also relates to adjuvant compositions comprising a novel polypeptide, rabbit GMCSF, for boosting antibody production in rabbits.

### BACKGROUND ART

The use of vaccines for the immunization of animals requires the reproducible production of protein antigens, which may be difficult and expensive. Whole, or usually, parts of the whole organism, toxin, or antigen is/are used for immunization. Vaccines require high or mostly, repeated doses of the antigen to be administered for maintaining a certain level of immunity in the host. Further, vaccine development is severely limited by the availability of useful antigens that can elicit a feasible response in an animal. The use of adjuvants for the enhancement of the immune response in an animal is well known in the art. A wide variety of adjuvants have been used, for example, mineral oil or oil containing adjuvants like complete Freund's adjuvant (FCA), incomplete Freund's adjuvant, Ribi adjuvant, Titermax, etc., adjuvants derived from bacteria like MDP (muramyl dipeptide), lipid A, lipopolysaccharide (LPS), etc., mineral compounds like aluminium phosphate, aluminium hydroxide and calcium phosphate, etc., liposomes, saponins complexed to membrane protein antigens (immune stimulating complexes), etc. Many adjuvants are toxic or cause mild to severe side effects, while some may only elicit a weak immune response in the host. Therefore, the development of safe and effective adjuvants is a continuing challenge. One newer approach in adjuvant development is the use of biological immunostimulatory adjuvants, like cytokines, as adjuvants.

Genetic or DNA immunization is known in the art and uses genes or the DNA encoding the antigenic protein of interest, rather than the polypeptides themselves, as the source of the immunogen. This is based on raw materials that is easy and inexpensive to manufacture, and can also be produced reproducibly. The host's cells take up the DNA that is introduced, and express the encoded antigen by normal cellular mechanisms. The antigen is then presented on the cell surface with host MHC class I and class II molecules where contact with immunocompetent cells evokes an immune response. Thus, without increasing the amount of antigen in the initial vaccine, or without repeated administration of the vaccine, immunity lasts for an extended period of time. Immunization of mice with naked DNA encoding a herpes simplex virus (HSV) protein was reported by Manickan et al., J Clin Invest 100: 2371-2375 (1997). Boyle et al., Proc Natl Acad Sci USA 94:14626-14631(1997) reported that DNA immunization induces rapid CTL responses, and produces higher avidity antibodies than traditional protein immunization. DNA vaccines for HIV/AIDS have been produced and tested in various animal models, including non-human primates, and in human clinical trials. See, e.g., Robinson et al., Ann N Y Acad Sci 772: 209-211 (1995); Yasutomi et al., J Virol 70:678-681 (1996); MacGregor et al., J Infect Dis 178: 92-100 (1998). In addition to infectious diseases, DNA immunization is believed to have the potential as a means for cancer immunotherapy (see, e.g., Srinivasan and Wolchok, J Transl Med 2: 12 (2004)). DNA vaccines have also been recommended for allergy treatment (see, e.g., Hartl et al., Methods 32: 328-39 (2004)).

Several factors influence the outcome of DNA vaccination, including, the method and location of immunization, the form of the immunogen, the immunization regimen, the presence or absence of adjuvants or the co-administration of biological adjuvants like cytokines, co-administration of other costimulatory molecules, the presence or absence of immunostimulatory sequences (ISS) within the DNA, etc. For example, it was observed that the DNA from bacteria, but not vertebrates, could induce a nonspecific immune response, which appeared to be due to differences in the frequency of unmethylated cytosine-phosphate-guanine dinucleotides (CpG) found in the two genomes. Further, it was found that DNA vaccination with a plasmid DNA containing the CpG and ISS sequences induced a more vigorous antibody and CTL response than an otherwise identical vaccine without the ISS sequence. Other important factors also influencing the immune response to DNA vaccination is the form of the encoded antigen, in particular, whether the antigen is expressed as a cytoplasmic or secreted protein, and the level of expression of the encoded antigen and/or adjuvants. In general, the higher and longer-lasting the expression, the more vigorous the immune response.

Successful DNA vaccination has been demonstrated via a number of different routes, including intravenous, intramuscular, intrasplenic and intrahepatic routes. For the generation of an antibody response, multiple studies have reported that gene gun immunization is far more efficient than needle injection, eliciting similar levels of antibody responses with 100- to 5000-fold less DNA. The optimal regimen for administering a DNA vaccine (e.g., dose, number, and/or frequency of immunizations) is far from determined and requires optimization for each individual antigen. Most studies indicate that multiple injections are necessary to maximize the immune response.

Of the different ways to modulate the immune response via DNA immunization, the most promising way may be through the coadministration of biological adjuvants such as cytokines. The GM-CSF gene is one of the most studied genetic adjuvants, and was shown to be an effective immune stimulator following DNA vaccination. Additional booster effects were observed when the GM-CSF gene was combined with other adjuvants like the FMS-like tyrosine kinase 3 ligand (Flt3L) gene or the IL-4 gene. Recently, the combination of GM-CSF and Flt3L was shown to result in high antibody titers in mice, in most (84%) of the more than 130 tested antigens; Chambers et al., Nat. Biotechnol. 21(9): 1088-92 (2003).

Even so, DNA immunization requires more effective methods that result in higher and longer-lasting expression of the encoded antigen proteins and/or adjuvants.

### SUMMARY OF THE INVENTION

The present invention concerns a method for DNA immunization or vaccination or stimulatation of the immune system of animals using recombinase/transposase mediated integration of expression cassettes encoding one or several antigens and/or adjuvants into the genome of transfected cells in vaccinated animals and also provides adjuvant compositions for boosting antibody production of DNA immunized animals.

In one aspect, the invention provides a novel adjuvant sequence of rabbit GMCSF polypeptide of SEQ ID NO: 1. Further, the invention provides a chimeric molecule comprising the rabbit GMCSF polypeptide of SEQ ID NO: 1 fused to a heterologous amino acid sequence. In one embodiment, the heterologous amino acid sequence is an epitope sequence. In another embodiment, the heterologous amino acid sequence is an immunoglobulin sequence. In a further aspect of this embodiment, the immunoglobulin sequence is an Fc region of an immunoglobulin.

The present invention also provides nucleotide sequences encoding the rabbit GMCSF polypeptide of SEQ ID NO: 1. Further, the invention provides a vector or an expression cassette comprising the nucleotide sequence(s) encoding the rabbit GMCSF polypeptide of SEQ ID NO: 1.

The present invention also provides an isolated host cell transformed with the nucleic acid sequences encoding the rabbit GMCSF polypeptide of SEQ ID NO: 1.

In another aspect, the invention concerns a method for immunizing or vaccinating an animal, comprising: (i) administering at least one DNA construct comprising at least one expression cassette that encodes for an antigen, and a first recombination site; (ii) stimulating the immune system of said animal by administering at least one adjuvant to said animal; and, (iii) administering a recombinase that mediates the integration of said expression cassette into the genome of said animal comprising a second recombination site, wherein, said antigen is expressed.

In all aspects of the invention, the adjuvant may be a traditionally used adjuvant or may be introduced as a polypeptide or as a nucleic acid encoding the adjuvant. When the adjuvant is introduced as a DNA, the method involves: (i) the administration of the adjuvant into the animal as a DNA construct comprising an expression cassette that encodes for the adjuvant, and, a first recombination site; and, (ii) a recombinase that mediates the integration of the expression cassette into the genome of the animal comprising a second recombination site, wherein, the adjuvant is expressed.

In all aspects, preferred antigen(s) include, but are not limited to, viral, bacterial, fungal, protozoal antigens and antigens associated with diseases like infection, inflammation, cancer, asthma/allergy, autoimmune diseases, multiple sclerosis, sepsis/toxic shock, rheumatoid arthritis, allograft rejections, psoriasis, etc. In a preferred embodiment, the antigen is CD20.

In all aspects, preferred adjuvant(s) include, but are not limited to, GMCSF, Flt3L, interleukins like IL-1α and β, IL-2, IL-12, IL-15, IL-8, IL-4, IL-5, IL-6, IL-10, TNF-α, TNF-β, IFN-γ, etc. and co-stimulatory molecules like TCA3, CD80 (B7.1), CD86 (B7.2), CD40 ligand (CD154), MCP-1, MIP-1α, β, RANTES, etc. In a preferred embodiment, the adjuvant is the rabbit GMCSF of SEQ ID NO: 1.

In one embodiment, the antigen-encoding expression cassette and the adjuvant-encoding expression cassette are part of one DNA construct. In another embodiment, the antigen-encoding expression cassette and the adjuvant-encoding expression cassette are on separate DNA constructs.

In all aspects of the invention, the recombinase may be administered as a polypeptide, or as an RNA molecule encoding the recombinase, or as a DNA construct comprising an expression cassette encoding the recombinase.

In one embodiment, the recombinase may be a site-specific recombinase expressed by a phage. In a further aspect of this embodiment, the phage recombinase may be selected from the group consisting of φC31, phage R4 and TP901-1. In another embodiment, the recombinase may be a site specific recombinase selected from the group consisting of a Cre-recombinase, a Cre-like recombinase, a Flp recombinase and an R recombinase.

In yet another embodiment, the recombinase may be a transposase or a retrotransposase. In a further aspect, the transposase is selected from the group consisting of AC7, Tn5, Tn916, Tn951, Tn1721, Tn2410, Tn1681, Tn1, Tn2, Tn3, Tn4, Tn5, Tn6, Tn9, Tn10, Tn30, Tn101, Tn903, Tn501, Tn1000, Tn1681, tn2901, AC transposons, Mp transposons, Spm transposons, En transposons, Dotted transposons, Mu transposons, Ds transposons, En transposons, and I transposons. In a yet another aspect, the transposase is a eukaryotic transposase.

In one embodiment, the first and second recombination sites share at least 90% sequence identity. In another embodiment, the first and second recombination sites share less than 90% sequence identity. In a further embodiment, the first recombination site comprises a bacterial genomic recombination site and the second recombination site comprises a phage recombination site. In yet another embodiment, the bacterial genomic recombination site is attB and the phage recombination site is attP. In yet another embodiment, the first recombination site comprises an attB site, and said second recombination site comprises a pseudo-attP site. In yet another embodiment, the first recombination site comprises a pseudo-attB site, and said second recombination site comprises an attP site.

In a certain embodiment, the recombinase-mediated recombination results in a site that is no longer a substrate for the recombinase.

In one embodiment of the invention, the DNA construct(s) may be circular. In another embodiment, the DNA construct(s)may be linear.

In a certain aspect, the invention provides a method for producing antibodies in an animal, comprising: (i) administering at least one DNA expression cassette encoding an antigen, a recombination site, and a recombinase that mediates the integration of the DNA expression cassette into the genome of the animal; (ii) optionally administering at least one adjuvant; (iii) harvesting a serum sample after several days from the animal; and (iv) identifying and optionally, purifying antibodies to the administered antigen(s) from the serum sample.

As indicated before, the adjuvant may be a traditionally used adjuvant or may be introduced as a polypeptide or as a nucleic acid encoding the adjuvant.

In all aspects, preferred animals include humans and non-human animals like rabbits, birds (e.g., chicken, turkey, ducks, geese, etc.), rodents, cows, pigs, sheep, goats and horses. In a preferred embodiment, the non-human animal is a rabbit.

A preferred group of non-human animals includes non-human transgenic animals carrying an exogenous immunoglobulin translocus. In one embodiment, the non-human transgenic animal is a gene converting animal. In a preferred embodiment, the exogenous immunoglobulin translocus is a human or humanized immunoglobulin heavy and/or light chain sequence.

### BRIEF DESCRIPTION OF THE DRAWINGS

FIG. 1 shows the amino acid sequence of rabbit GMCSF (SEQ ID NO:1).

FIG. 2 shows a nucleic acid sequence (SEQ ID NO:2) encoding the rabbit GMCSF polypeptide. The coding sequence is highlighted.

FIG. 3 shows an amino acid alignment of rabbit GMCSF (SEQ ID NO:1) with other GMCSF molecules (SEQ ID NOs: 7-19) derived from various animals.

FIG. 4 shows an expression plasmid with three expression cassettes encoding rabbit GM-CSF, rabbit FLT3-L and human CD20. The plasmid also contains a recombinase recognition sequence (RRS) for recombinase-mediated integration of the expression cassettes into the genome of transfected cells.

### DETAILED DESCRIPTION OF THE INVENTION

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994); Lowrie and Whalen, DNA Vaccines: Methods and Protocols, Humana Press (1999); Constantin A. Bona and Adrian Bot, Genetic Immunization, Kluwer Academic/Plenum Publishers (New York, N.Y. 2000); Koprowski and Weiner, DNA Vaccination/Genetic Vaccination, Springer (Berlin, N.Y., 1998); “Molecular Cloning: A Laboratory Manual”, 2^{nd} edition (Sambrook et al., 1989); “Oligonucleotide Synthesis” (M. J. Gait, ed., 1984); “Gene Transfer Vectors for Mammalian Cells” (J. M. Miller & M. P. Calos, eds., 1987); and “Current Protocols in Molecular Biology” (F. M. Ausubel et al., eds., 1987) provide one skilled in the art with a general guide to many of the terms, methods and protocols used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “immunization” is used in the broadest sense and refers to the introduction of antigens into the body in order to stimulate the development of immunity. “DNA immunization” or “genetic immunization” uses DNA encoding one or more antigens and/or adjuvants, instead of the proteins/polypeptides themselves, to produce and/or boost immunity.

The term “vaccination” usually refers to the introduction of a killed or attenuated pathogen into the body in order to promote protective immunity.

In “DNA vaccination” (also known as “genetic vaccination”), instead of the killed or attenuated pathogen, one or more genes of one pathogen or different pathogens is/are introduced into the body. As a result, it is possible to vaccinate against variants of the same pathogen, or against several different pathogens, at the same time.

The term “DNA construct” as used herein refers to a polynucleotide molecule, which contains one or several structural gene(s) of interest, recombination sequences and other DNA sequences necessary for maintenance, replication, selection of the DNA construct. A DNA construct may contain one or more of the “expression cassettes” described below. A DNA construct can be any vector, like a plasmid, any viral vector including, but not limited to, retroviral, adenoviral, lentiviral, a cosmid, etc. The term “retroviral vector” also refers to a DNA construct and refers to a retrovirus or retroviral particle, which is capable of entering a cell and integrating the retroviral genome into the genome of the host cell. The DNA construct can be either linear, or preferably, circular.

The term “expression cassette” refers to a polynucleotide molecule, which contains one or several structural gene(s) of interest operatively linked to their respective regulatory sequences that promote expression of the encoded gene of interest, and optionally, further DNA sequences that encode for domains required for various functions such as, for timely expression, to facilitate proper protein folding, for uptake by antigen presenting cells, for B-cell activation, T-helper cell recognition etc. In the context of this invention, expression cassettes include one or more antigen expression cassettes, adjuvant expression cassettes, recombinase expression cassettes and the like.

The term “recombinase” as used herein refers to a group of enzymes that can facilitate recombination, preferably, site specific recombination, between defined sites, called “recombination sites,” where the two recombination sites are physically separated within a single nucleic acid molecule or on separate nucleic acid molecules. The sequences of the two defined recombination sites are not necessarily identical. Within the group of recombinases there are several subfamilies including “integrases” (for example, like Cre, Cre-like, FLP and λ integrase), “resolvases/invertases” (for example, φC31 integrase, R4 integrase, and TP-901 integrase). The term “recombinase” also includes, but is not limited to, prokaryotic or eukaryotic transposases, viral or Drosophila copia-like or non-viral reterotransposons that include mammalian reterotransposons. Exemplary prokaryotic transposases include transposases encoded in the transposable elements of Tn1, Tn2, Tn3, Tn4, Tn5, Tn6, Tn9, Tn10, Tn30, Tn101, Tn501, Tn903, Tn1000, Tn1681, Tn2901, etc. Eukaryotic transposases include transposases encoded in the transposable elements of Drosophila mariner, sleeping beauty transposase, Drosophila P element, maize Ac and Ds elements, etc. Retrotransposases include those encoded in the elements of L1, Tol2 Tc1, Tc3, Mariner (Himar 1), Mariner (mos 1), Minos, etc. Transposases may also be selected from Mp, Spm, En, dotted, Mu, and I transposing elements.

The term “wild-type recombination site” as used herein refers to a recombination site normally used by a recombinase, such as an integrase.

By “pseudo-recombination site” is meant a site at which recombinase can facilitate recombination even though the site may not have a sequence identical to the sequence of its wild-type recombination site.

In the context of the present invention, the terms “first recombination site” or “second recombination site” can be any wild-type or pseudo-recombination site, such as, for example, an attB, attP, pseudo-attB, or a pseudo-attP site.

The term “recombinase-mediated integration of the expression cassette” is used to refer to integration mediated by an encoded and expressed recombinase, that facilitates specific integration of the expression cassette into the genome of a cell rather than random integration.

“Adjuvant” is any compound or composition whose purpose is to enhance the immune response to a particular antigen of interest. Any adjuvant, irrespective of the mechanism by which it enhances the immune response, is useful in this invention.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

The term “non-human animal” as used herein includes, but is not limited to, mammals such as, for example, non-human primates, rodents (e.g., mice and rats), and non-rodent animals, such as, for example, rabbits, pigs, sheep, goats, cows, pigs, horses and donkeys. It also includes birds (e.g., chickens, turkeys, ducks, geese and the like). The term “non-primate animal” as used herein refers to mammals other than primates, including but not limited to the mammals specifically listed above.

The terms “polynucleotide” and “nucleic acid” are used interchangeably, and, when used in singular or plural, generally refer to any polyribonucleotide or polydeoxribonucleotide, which may be unmodified RNA or DNA or modified RNA or DNA. The DNA origin maybe from genomic DNA, cDNA or through gene synthesis. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

B. Detailed Description

The present invention concerns an improved method for the DNA immunization or vaccination of animals such as mammals, including humans. The method uses DNA expression cassettes encoding an antigen (such as a protein from a pathogen or parts thereof, a tumor antigen, etc.), rather than the antigen itself, in order to produce long-lasting immunity. The improved method can also be used to enhance or stimulate immunity in an animal using genetic adjuvants, that is, adjuvants encoded for by a DNA expression cassette, for longer lasting enhancement of immunity. In this method, the DNA encoding the antigen or the adjuvant is introduced and subsequently, integrated into the genome of the animal via a recombinase, preferably a site-specific recombinase, that facilitates this integration into the genome. This invention further provides novel nucleic acid and polypeptide sequences for the rabbit GMCSF adjuvant. Thus, for example, antibodies, including humanized antibodies, can be generated in animals like rabbits, using this adjuvant.

More specifically, site-specific integration of the antigen and/or adjuvant expression cassettes comprises administration of: (i) one or several circular DNA constructs comprising one or several antigen and/or adjuvant encoding expression cassettes and a first recombination site recognized by a site-specific recombinase, and (ii) a site specific recombinase. Administration of the DNA constructs into the animal using any of the methods discussed below, results in the transfection of cells. The genome of transfected cells comprise a second recombination site native to the genome, and thus, site-specific recombination between the first and the second recombination sites facilitated by the recombinase results in stable and a higher incidence of integration of the DNA expression cassette(s) into the cellular genome of the animal. Thus, recombinase-mediated DNA immunization method results in higher and longer-lasting expression of the encoded antigenic and/or adjuvant protein(s).

In one aspect of the invention, the methods comprise DNA that encodes for antigens. The origin of the antigenic DNA includes, but is not limited to, genomic DNA, cDNA or sequences obtained by gene synthesis. Antigens may be identified by genome-wide searches for novel useful sequences using strategies known in the art or by bioinformatic screens.

By antigenic DNA is meant DNA sequences from an infectious pathogen including, but are not limited to, bacteria, virus, protozoa, Clamydia, Leishmania, Toxoplasma, Plasmodium, fungus including yeast, etc. or parts of the antigen thereof.

Exemplary bacterial antigens, which are encoded for by their DNA in the present invention include, antigens from Staphylococcus aureus, recombinant versions of virulence factors such as alpha-toxin, adhesin binding proteins, collagen binding proteins, and fibronectin binding proteins. Exemplary bacterial antigens also include other proteins of S. aureus, Pseudomonas aeruginosa, enterococcus, enterobacter, and Klebsiella pneumoniae, Bordetella (cya C and cya A genes), etc. Further exemplary bacterial antigens include, but are not limited to, the coding sequences of capsular antigens, recombinant versions of outer membrane proteins, fibronectin binding proteins, antigens and toxins from Pseudomonas aeruginosa, enterococcus, enterobacter, Klebsiella pneumoniae, etc.

Exemplary antigens for the generation of antibodies against fungi include outer membrane proteins of fungi, such as, for example, Candida albicans, Candida parapsilosis, Candida tropicalis, and Cryptococcus neoformans, etc.

Exemplary antigens, the coding sequences of which can be used to generate antibodies against viruses include, but are not limited to, the envelop proteins and attenuated versions of viruses which include, but are not limited to, influenza, HIV-½ (especially, gag, pol, rev, nef and envelope proteins like gp120, env, etc.), rabies, respiratory syncitial virus (RSV) (particularly the F-Protein), Hepatitis C virus (HCV), Hepatitis B virus (HBV), cytomegalovirus (CMV), EBV, rotavirus, Ebola and HSV-1 and 2 (Herpes simplex virus), etc.

In another aspect of the invention, by antigen is also meant antigens that elicit antibody responses, wherein the antibody is useful for the therapeutic treatment of diseases like cancer. Exemplary cancer related antigens useful in the preparation of therapeutic antibodies include, but are not limited to, carcinoembryonic antigen (CEA) and 17-1A for colon cancer; T cell receptor Vβ for cutaneous T cell lymphoma; Her-2-neu antigen for breast cancer; CD19, CD20, CD22 and CD53 antigens for B cell lymphomas; prostate specific membrane antigen (PMSA) for prostate cancer; VEGF (general); CA125 for ovarian cancer; EpCAM for colorectal cancer, etc.

By antigen is further meant antigens eliciting antibody responses, wherein the antibody is useful for the therapeutic treatment of diseases other than cancer, including, but not limited to, asthma/allergies with exemplary antigens like CD23, IgE, IL-5, IL-4, etc.; autoimmune diseases with exemplary antigens like glycosyl CD3 (for Type I diabetes), CD3, CD4, CD40L (for SLE or lupus), etc.; multiple sclerosis with exemplary antigens like VLA-4, CD40L, CD11/18, etc.; inflammation and/or sepsis/toxic shock with exemplary antigens like TNFα and β, CD14, etc.; rheumatoid arthritis with exemplary antigens like complement C5, TNFα and β, CD4, etc.; allograft rejections with exemplary antigens like CD147, CD18, CD40L, β2 integrin, CD3, CD4, CD25, etc.; psoriasis with exemplary antigens like IL-8, CD11a, E-selectin, ICAM-3, CD80, CD2, CD3; wherein, such resulting immune responses may be used in the preparation of immunovaccines to combat the disease.

By antigen is also meant antigens that elicit an antibody response, wherein the antibody is an agonistic or a mimetic antibody useful for treating a disease, for example, like thrombocytopenia. Here, the mimetic antibody, anti-c-MPL, is designed to mimic the activity of TPO (thrombopoietin) that is responsible for platelet production and hence, is useful as a therapeutic antibody.

In yet another aspect of the invention, the method for DNA immunization uses an adjuvant. Adjuvants of the present invention include, but are not limited to, traditionally used adjuvants like mineral oil or oil containing adjuvants like complete Freund's adjuvant (FCA), incomplete Freund's adjuvant, Ribi adjuvant, Titermax, etc., adjuvants derived from bacteria like MDP (muramyl dipeptide), lipid A, lipopolysaccharide (LPS), etc., mineral compounds like aluminium phosphate, aluminium hydroxide and calcium phosphate, etc., liposomes, saponins complexed to membrane protein antigens (immune stimulating complexes), cytokines, co-stimulatory molecules, bacterial DNA, CpG, etc.

In one embodiment, the adjuvant of the present invention is any cytokine like GMCSF, IL-1α and β, IL-2, IL-12, IL-15, IL-18, IL-4, IL-5, IL-6, IL-10, TNF-α, TNF-β, IFN-γ, or the like. In another embodiment, the adjuvant is a co-stimulatory molecule like TCA3, CD80 (B7.1), CD86 (B7.2), CD40 ligand (CD154), MCP-1, MIP-1α, β, RANTES, or the like.

In another embodiment of the present invention, the cytokine or co-stimulatory adjuvant maybe introduced as a polypeptide, as an mRNA or as a DNA encoding the adjuvant or co-stimulatory molecule. Further, a combination of adjuvants or co-delivery of more than one adjuvant may be also be used. Ideally, the synergy between the combination of adjuvants being used is evaluated and their combined ability to elicit both, humoral and cell-mediated immune responses, even if administered by various routes.

In an important aspect of the invention, the methods utilize a recombinase to facilitate integration of the antigenic and/or adjuvant expression cassette(s), or even the recombinase expression cassette, into the genome of the animal. Genomic integration of expression cassettes may be facilitated by site-specific or random recombination. In a preferred embodiment, the method requires a site-specific recombinase that facilitates genomic integration of any of the expression cassettes at a specific site into the genome of the animal.

Site specific recombinases are enzymatically active proteins that catalyze a reciprocal double-stranded DNA exchange between two DNA segments. Such recombinases recognize specific sequences in both partners of the exchange and may function as sole proteins, or may require the presence of accessory factors for function. While the mechanism of catalysis might be different for different types of site-specific recombinases, they are all included herein, regardless of the underlying mechanism, and are suitable for the practice of the present invention.

Site-specific recombinases are typically, but not exclusively, prokaryotic. The two largest families of site-specific recombinases are λ integrase-like enzymes and the resolvase/invertases. Members of the two families significantly differ in their amino acid sequences, and in their mechanisms of catalysis. Recombination by members of the λ integrase family involves the formation and resolution of a Holliday junction intermediate during which the DNA is transiently attached to the enzyme through a phosphotyrosine linkage. The resolvase/invertase family of enzymes act via a concerted, four-strand staggered break and rejoining mechanism during which a phosphoserine linkage is formed between the enzyme and the DNA.

Thus, for example, the genome of the broad host range Streptomyces temperate phage, ΦDC31 is known to integrate into the host chromosome with the aid of an enzyme that is a member of the resolvase/invertase family of site-specific recombinases. For further details see, e.g., Thorpe and Smith, Proc. Natl. Acad. Sci. USA, 95(10):5505-5510 (1998). The phage C31 integrase has been shown to mediate efficient integration in the human cell environment at attB and attP phage attachment sites on extrachromosomal vectors. φC31 and R4 belong in the integrase family of site-specific recombinases, while TP901-1 belongs to the family of extended resolvases. The R4 integrase is a site-specific, unidirectional recombinase derived from the genome of phage R4 of Streptomyces parvulus. The site-specific integrase TP901-1 is encoded by phage TP901-1 of Lactococcus lactis subsp. cremoris. λ is a temperate bacteriophage that infects E. coli. The phage has one attachment site for recombination (attP) and the E. coli bacterial genome has an attachment site for recombination (attB). In the context of the present invention, wild-type recombination sites can be derived, for example, from the homologous system and associated with heterologous sequences. Thus, the attB site can be placed in other systems to act as a substrate for an integrase. In one embodiment, the recombinase may catalyze recombination between a bacterial genomic recombination site (attB) and a phage genomic recombination site (attP), or the first site may comprises a pseudo-attB site and/or the second site may comprises a pseudo-attP site, or vice versa. In another embodiment, the recombinase mediates production of recombination sites that are no longer substrates for the recombinase (Groth et al., Proc. Nat. Acad. Sci., 2000, 97: 5995-6000; Olivares et al., Nature Biotechnol. 2002, 20(11):1124-8); (Thyagarajan et al., Mol. and Cell. Biol., 2001, 21: 3926-3934); Hollis et al., Repro. Biol. and Endocrinol., 2003, 1:79. Thus, in the present invention, the recombinase may be a site-specific recombinase encoded by a phage selected from the group consisting of λ integrase, φC31, TP901-1, and R4. Other known and frequently used site-specific recombinases include Cre and FLP or the like (see, e.g., Bouhassira et al., Blood 88 (Suppl. 1), 190a (1996); Bouhassira et al., Blood 90:3332-3344 (1997); Seibler & Bode, Biochemistry 36:1740-1747 (1997); Seibler et al., Biochemistry 37:6229-6234 (1998); Bethke & Sauer, Nucl. Acids Res. 25:2828-2834 (1997)). The target of the Cre recombinase is a 34-bp sequence loxP sites that consists of two inverted 13-bp Cre-binding sites separated by an eight base spacer within which the recombination occurs (Hoess & Abremski, Proc. Natl. Acad. Sci. USA 81:1026-1029 (1984)). Cre/loxP based cloning systems are commercially available, for example, from BD Biosciences-Clontech, Palo Alto, Calif. (Creator™), or Invitrogen, Carlsbad, Calif. (Echo™). Flp targets the frt site. The use of Cre recombinase for site-specific recombination of DNA in eukaryotic cells is described in U.S. Pat. No. 4,959,317. The use of site specific recombinase for the transfection of eukaryotic cells is described in U.S. Pat. No. 6,632,672. Site specific recombination in general is described in U.S. Pat. No. 4,673,640.

In yet another embodiment, the recombinase can be a transposase or a retrotransposase. Transposons or retrotransposases, are enzymes that catalyze their transposition by a cut and paste mechanism and thus, can be used for the transfer or insertion of any expression cassette. They provide non-viral and non-homologous methods for the insertion or transfer of any DNA sequence into the genomes of a wide range of species, including vertebrates like humans, bird, rodents, etc. For example, the Drosophila element mariner was shown to transpose itself into chicken germ lines, Sherman et al., Nature Biotechnol., 16:1050-1053 (1998). Long term transgene expression or efficient insertion of transposon DNA, using the sleeping beauty transposase system into mammalian systems like the mouse and human genomes have been demonstrated by Yant et al., Nature Genetics, 25: 35-41 (2000); Dupuy et al., Proc. Nat. Acad. Sci., 99: 4495-4499 (2002) and Geurts et al., Mol. Therapy, 8: 108-117 (2003). Other transposes like L1, Tol2 Tc1, Tc3, Mariner (Himar 1), Mariner (mos 1), Minos have been shown to be active in vertebrate species and are thus useful for gene transfer or as insertional mutagenesis vectors, Largaespada, David A., Repro. Biol. and Endocrinol., 1:80 (2003). Exemplary transposases include, but are not limited to, prokaryotic or eukaryotic transposases, viral, Drosophila copia-like or non-viral reterotransposons which include mammalian reterotransposons, etc. Prokaryotic transposases include transposases encoded in the transposable elements of Tn1, Tn2, Tn3, Tn4, Tn5, Tn6, Tn9, Tn10, Tn30, Tn101, Tn501, Tn903, Tn1000, Tn1681, Tn2901, etc. Eukaryotic transposases include transposases encoded in the transposable elements of Drosophila mariner, sleeping beauty transposase, Drosophila P element, maize Ac and Ds elements, etc. Retrotransposases include those encoded in the elements of L1, Tol2 Tc1, Tc3, Mariner (Himar 1), Mariner (mos 1), Minos, etc. Transposases may also be selected from Mp, Spm, En, dotted, Mu, and I transposing elements.

In a certain embodiment, the transposase may be a transposase selected from the group of AC7, Tn5, Tn916, Tn951, Tn1721, In2410, Tn1681, Tn1, Tn2, Tn3, Tn4, Tn5, Tn6, Tn9, Tn10, Tn30, Tn101, Tn903, Tn501, Tn1000, Tn1681, tn2901, AC transposons, Mp transposons, Spm transposons, En transposons, Dotted transposons, Mu transposons, Ds transposons, En transposons, I transposons and the like. Alternatively, an altered target site transposase or eukaryotic transposase, like Drosophila P element, Drosophila mariner element, or sleeping beauty transposase, and the like may be used.

In yet another embodiment, multiple copies of expression cassettes encoding antigen(s) and/or adjuvant(s) may be inserted into the genome of a eukaryotic cell by a “rolling replication” transposition. Tn1, Tn2, Tn3, Tn4, Tn5, Tn9, Tn21, Tn501, Tn551, Tn951, Tn1721, Tn2410 and Tn2603 are examples of rolling replication type transposons.

The recombinase may be introduced as an enzymatically active protein or in form of a recombinant expression plasmid encoding the recombinase. Alternatively, the expression of recombinase may be accomplished through introduction of messenger RNA encoding recombinase.

Thus, the invention concerns a method of DNA vaccination including transposase-mediated integration of expression cassettes encoding one or several antigens and/or adjuvants into the genome of transfected cells of vaccinated animals.

According to the present invention, the nucleic acids encoding the antigen, the recombinase and the adjuvant may be added simultaneously or in any order. For example, the DNA encoding the antigen and the adjuvant may be added prior to the addition of the recombinase. Alternately, the recombinase may be introduced into the recipient cell before or concurrent with the introduction of the expression cassette comprising the coding sequence of the desired antigen or antigens and/or the coding sequence of the desired adjuvant(s) as well as the recombinase-specific recognition sequence. Or, the nucleic acid(s) encoding the antigen, the recombinase and the recombinase-specific recognition sequence may be added first and the adjuvant, or the DNA encoding it may be added subsequently.

In one embodiment, the recombinase is introduced into the cell as a mRNA, e.g., by injection into male pronuclei with the aid of a micromanipulator. Alternatively, the recombinase may be introduced into the recipient cell by a recombinant expression cassette (e.g., plasmid) encoding the recombinase. Such plasmids are known in the art and are either commercially available or can be readily made, and include the commercially available expression plasmid, pcDNA3, and its variants. Cre expression plasmids are also commercially available, and include, for example pBS 185 (CMV-CRE) (Clontech). In a further embodiment, the recombinase is introduced into the recipient animal as an enzymatically active protein.

In the present invention, the DNA construct can be any vector like a plasmid, any viral vector, including, but not limited to, retroviral, adenoviral, lentiviral, cosmid, phage, etc. Further, the DNA construct can be either linear, or preferably, circular. The DNA constructs of the present invention can encode for the antigenic DNA, the adjuvant DNA and/or the recombinase DNA on one construct, one two or more constructs, or on separate individual constructs. Each DNA construct may be simultaneously administered to the animal or, preferably, the adjuvant construct may be administered after the introduction and expression of the antigenic DNA expression cassette. The DNA constructs of the invention may further include DNA sequences necessary, for example, for its maintenance, replication, antibiotic selection, etc. Further, the DNA constructs may encode for various functional DNA sequences, for example, immunostimulatory (ISS) sequences like CpG motifs, that trigger a more vigorous immune response, etc.

Routes of administration of the DNA constructs of the invention include injection, oral or intranasal delivery, etc, or by any other route known in the art or as described, for example, in U.S. Pat. No. 5,543,158, U.S. Pat. No. 5,641,515, and U.S. Pat. No. 5,399,363, all of which have been hereby incorporated by reference and is regardless of the mechanism by which they elicit an immune response. Injection can be subcutaneous, intradermal, intramuscula, intraperitoneal, intrasplenic, transdermal, etc. A preferred method for injecting the DNA constructs of the invention is via a gene gun.

The DNA can be injected into the animal as naked DNA, with lipofection reagents, with carriers, or with any composition known to enhance the uptake of DNA by the cell, as described for example in U.S. Pat. Nos. 4,608,251; 4,601,903; 4,599,231; 4,599,230; 4,596,792; and 4,578,770, all of which have been hereby incorporated by reference. A carrier includes any other active ingredient required for the working of the vaccine composition, any solvent, dispersion media, vehicle, coating, diluent, anti-bacterial or anti-fungal agent, buffer, isotonic solution, absorption delaying agent, colloid, suspension medium, etc.

The effective amount of the DNA construct(s) to be injected varies depending on, for example, the type of antigen and its level of expression (needs to be standardized for each antigen), the route of administration, size (body weight of the host), form of the encoded antigen, that is, whether the antigen is a cytoplasmic, membrane bound or a secreted protein, etc. The effective amount or dosage of a DNA construct to be injected into an animal is that amount of DNA that effectively achieves optimal immunization or immunotherapeutic treatment for any administered antigen. For example, an injection dosage of approx. 1 μg of DNA per Helios gene gun bullet per animal was found to be optimal for some antigens used in this invention but any other suitable dosages may also be used. The DNA immunization may be done repeatedly, according to a suitable schedule, depending on the type of disease being treated, the dosage required to maintain a protective level of immunity, etc. as can be routinely determined by one skilled in the art.

Besides coding for structural DNA sequences of interest, expression cassettes may further contain sequences encoding for secretion leader sequences to ensure secretion of the encoded antigen, domains that improve protein solubility and/or protein folding to enhance uptake of the antigen by antigen presenting cells (e.g., the 46 residue COMP domain), epitopes for T-helper-independent B-cell activation, etc.

The present invention further provides nucleic acid sequences that encode for proteins, polypeptides or peptide sequences for rabbit GMCSF, which are useful as an adjuvants. Expression of GMCSF may be under the control of a constitutively active promoter, a tissue specific promoter or an inducible promoter.

It is also contemplated that a given nucleic acid sequence for rabbit GMCSF may be represented by natural variants that have slightly different nucleic acid sequences but, nonetheless, encode the same protein. Furthermore, the term functionally equivalent codon is used herein to refer to codons that encode the same amino acid, such as the six codons for arginine or serine (Table 1), and also refers to codons that encode biologically equivalent amino acids, as discussed herein.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The DNA segments used in the present invention encompass biologically functional equivalent modified polypeptides and peptides. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to introduce improvements to the antigenicity of the protein, to reduce toxicity effects of the protein in vivo to a subject given the protein, or to increase the efficacy of any treatment involving the protein.

Allowing for the degeneracy of the genetic code (Table 1), the invention encompasses sequences that have at least about 50%, usually at least about 60%, more usually about 70%, most usually about 80%, preferably at least about 90% and most preferably about 95% of nucleotides that are identical to the nucleotides of SEQ ID NO: 2.

The term biologically functional equivalent is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of a rabbit GMCSF polypeptide, provided the biological activity of the protein is maintained.

The term functionally equivalent codon is used herein to refer to codons that encode the same amino acid, such as the six codons for arginine or serine, and also refers to codons that encode biologically equivalent amino acids (Table 1).

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, as discussed below. Table 1 shows the codons that encode particular amino acids.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0.+−0.1); glutamate (+3.0.+−0.1); serine (+0.3); asparagine (+0.2) glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within .+−0.2 is preferred, those that are within .+−0.1 are particularly preferred, and those within .+−0.0.5 are even more particularly preferred.

As outlined herein, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure (Johnson 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of adjuvants with altered and improved characteristics.

Thus, variant nucleic acid sequences that encode for rabbit GMCSF and functionally equivalent polypeptides of rabbit GMCSF are useful as adjuvants in this invention.

In another aspect of this invention, the animals to whom the DNA constructs of the invention can be administered include, but are not limited to, mammals (e.g., humans, non-human primates, rodents (e.g., mice and rats), non-rodents (e.g., rabbits, pigs, sheep, goats, cows, pigs, horses and donkeys), and birds (e.g., chickens, turkeys, ducks, geese and the like). The animals to whom the DNA constructs of the invention can be administered include ‘gene converting animals’, that is, animals that create antibody diversity substantially by gene conversion and/or somatic hypermutation (for e.g., rabbits, birds, cows, swine, etc.), and animals where antibody rearrangement stops early in life, that is, typically, within the first month of life (for e.g., rabbits, birds, sheep, goats, cattle, swine, horses, etc.)

Further, animals to whom the DNA constructs of the invention can be administered also include any of the non-human animal described above, further carrying a transgene encoding an exogenous immunoglobulin translocus, preferably, a human or humanized immunoglobulin heavy chain and/or immunoglobulin light chain sequence or parts thereof. The transgene locus can be either in the germline configuration or in a rearranged form. Since the transgenes encode for human or humanized immunolobulins or parts thereof, it results in the generation of humanized antibodies. Thus, for example, using the recombinase mediated DNA immunization methods described above, antibodies, including humanized antibodies, can be generated in target non-human animals using the rabbit GMCSF adjuvant described in this invention.

The invention is further illustrated by, but by no means limited to, certain embodiments referenced in the examples below. One skilled in the art will understand that various modifications of the present invention can be performed without substantial change in the way the invention works. All of such modifications are specifically intended to be within the scope of the invention claimed herein.

### EXAMPLE 1

### Cloning of Rabbit GMCSF

Peritoneal macrophages from ZIKA rabbits were harvested by lavage of the peritoneal cavity with 30-60 ml PBS/2% FCS (Gibco; PET 10270098), and washed twice. The cells were counted and plated in one well of a 24 well plate with a density of 1-3×10⁵ cells/well in DMEM/10% FCS and stimulated for 5 hrs with 1 μg/ml LPS E. coli 0111:B4 (Sigma; L2630) in a humidified 5% CO₂ incubator at 37° C. Cells were harvested and total RNA was isolated with QIA_{Amp} RNA-Mini-Kit (Qiagen) according to the manufactures instructions.

3-6 μl total RNA was reverse transcribed with the 3′-RACE CDS Primer A (5′-AAGCAGTGGTATCAACGCAGAGTAC(T)₃₀VN-3′) (SEQ ID NO: 3) using the BD SMART RACE cDNA Amplification Kit (BD Biosciences) according to the manufactures protocol. Rabbit GMCSF was PCR amplified from first strand cDNA with the primer pair GMCSFup3 (5′-aaggctaaggtcctgaggagg-3′) (SEQ ID NO: 4) and 10× universial primer A mix (mixture of

5′CTAATACGACTCACTATAGGGCAAGCAGTGGTATCAACGCAGAGT-3′ (SEQ ID NO: 5) and 5′-CTAATACGACTCACTATAGGGC-3′) (SEQ ID NO: 6) using the Eppendorf Triple Master polymerase with High Fidelity buffer. PCR conditions were: Denaturation at 94° C. for 50 s, annealing at 60° C. for 50 s and extension at 72° C. for 50 s, 35 cycles. PCR products were analysed by electrophoresis in 1.2% agarose gels. Specific bands were cut and extracted using the Gene Clean Turbo gel extraction Kit (Bio101) and cloned into TOPO TA Cloning Vector (Invitrogen) and sequenced (Agowa).

### EXAMPLE 2

### Generation of anti-CD20 Antibodies in Rabbits by Genetic Immunization

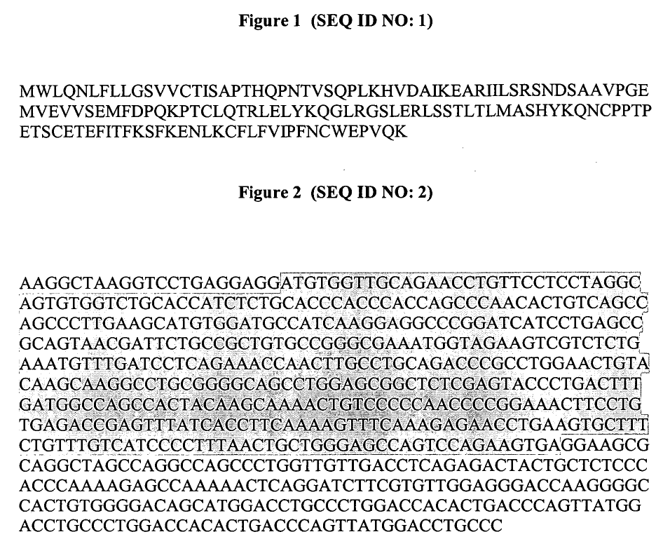

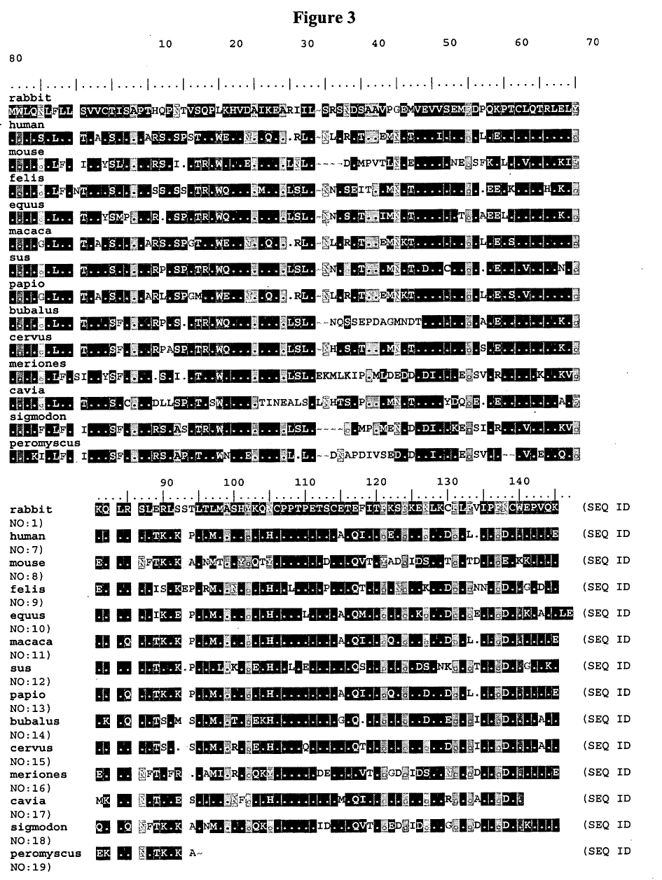

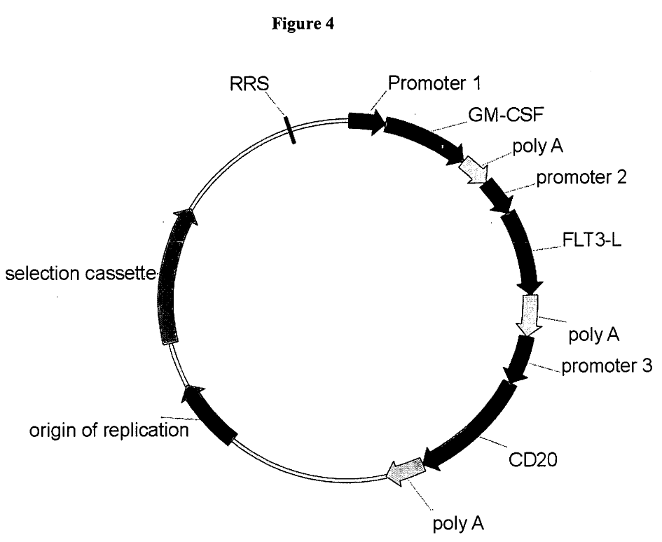

Messenger RNA (mRNA) from rabbit LPS-stimulated peritoneal macrophages and lymphocytes is isolated and reverse transcribed. Rabbit GMCSF and Flt3L cDNAs are amplified by PCR. Subsequently, the cDNAs are cloned into an expression plasmid. A cDNA encoding the soluble part of human CD20 is synthesized and cloned into the same expression plasmid. The final construct is shown in FIG. 1. Plasmid DNA is purified and mixed with an equal amount of a plasmid encoding C31 integrase. Plasmid DNA (about 1 μg/bullet) is coated on Helios gene gun bullets according to the manufacturer's instructions. Rabbits are mildly anesthetized and shot in each ear on day 0 and day 14. Blood is collected and allowed to coagulate. Sera are collected by centrifugation. Anti-CD20 antibodies are detected by flow cytometry using human peripheral blood lymphocytes. A comparison of animals immunized with and without rabbit GMCSF shows that administration of rabbit GMCSF increases the immune response significantly.

What is claimed is:

1. A rabbit GMCSF polypeptide of SEQ ID NO: 1.

2. A chimeric molecule comprising the polypeptide of claim 1 fused to a heterologous amino acid sequence.

3. The chimeric molecule of claim 2 wherein said heterologous amino acid sequence is an epitope sequence.

4. The chimeric molecule of claim 2 wherein said heterologous amino acid sequence is an immunoglobulin sequence.

5. The chimeric molecule of claim 4 wherein said immunoglobulin sequence is an Fc region of an immunoglobulin.

6. A nucleotide sequence comprising a nucleotide sequence encoding the rabbit GMCSF polypeptide of claim 1.

7. A vector or expression cassette comprising the nucleotide sequence of claim 6.

8. An isolated host cell transformed with the nucleic acid of claim 6.

9. A method for immunizing or vaccinating an animal, said method comprising:
(i) administering at least one DNA construct comprising at least one expression cassette that encodes for an antigen, and a first recombination site;
(ii) stimulating the immune system of said animal by administering at least one adjuvant to said animal; and,
(iii) administering a recombinase that mediates the integration of said expression cassette into the genome of said animal comprising a second recombination site,
wherein, said antigen is expressed.

10. The method of claim 9 wherein said adjuvant is introduced as a polypeptide.

11. The method of claim 9 wherein the administration of the adjuvant comprises introducing to said animal: (i) said adjuvant as a DNA construct comprising an expression cassette that encodes for said adjuvant, and, a first recombination site; and, (ii) a recombinase that mediates the integration of said expression cassette into the genome of said animal comprising a second recombination site, wherein, said adjuvant is expressed.

12. The method of claim 9 wherein said antigen(s) is/are selected from the group consisting of viral, bacterial, fungal, protozoal antigens and antigens associated with diseases like infection, inflammation, cancer, asthma/allergy, autoimmune diseases, multiple sclerosis, sepsis/toxic shock, rheumatoid arthritis, allograft rejections, psoriasis, etc.

13. The method of claim 9 wherein said antigen is CD20.

14. The method of claim 11 wherein said adjuvant(s) is/are selected from the group consisting of GMCSF, Flt3L, interleukins like L-1α and β, IL-2, IL-12, IL-15, IL-18, IL-4, IL-5, IL-6, IL-10, TNF-α, TNF-β, IFN-γ, etc. and co-stimulatory molecules like TCA3, CD80 (B7.1), CD86 (B7.2), CD40 ligand (CD154), MCP-1, MIP-1α, β, RANTES, etc.

15. The method of claim 11 wherein said adjuvant is the rabbit GMCSF of SEQ ID NO:1.

16. The method of claim 11 wherein said antigen-encoding expression cassette and said adjuvant-encoding expression cassette are part of one DNA construct.

17. The method of claim 11 wherein said antigen-encoding expression cassette and said adjuvant-encoding expression cassette are on separate DNA constructs.

18. The method of claim 9 or 11 wherein said recombinase is administered as a polypeptide.

19. The method of claim 9 or 11 wherein said recombinase is administered as a messenger RNA molecule encoding said recombinase.

20. The method of claim 9 or 11 wherein said recombinase is administered as a DNA construct comprising an expression cassette encoding said recombinase.

21. The method of claim 9 or 11 wherein said recombinase is a site-specific recombinase expressed by a phage.

22. The method of claim 21 wherein said recombinase is selected from the group consisting of φC31, phage R4 and TP901-1.

23. The method of claim 21 wherein said site specific recombinase is selected from the group consisting of a Cre-recombinase, a Cre-like recombinase, a Flp recombinase and an R recombinase.

24. The method of claim 9 or 11 wherein said recombinase is a transposase or a retrotransposase.

25. The method of claim 24, wherein said transposase is selected from the group consisting of AC7, Tn5, Tn916, Tn951, Tn1721, Tn2410, Tn1681, Tn1, Tn2, Tn3, Tn4, Tn5, Tn6, Tn9, Tn10, Tn30, Tn101, Tn903, Tn501, Tn1000, Tn1681, tn2901, AC transposons, Mp transposons, Spm transposons, En transposons, Dotted transposons, Mu transposons, Ds transposons, En transposons, and I transposons.

26. The method of claim 24, wherein said transposase is a eukaryotic transposase.

27. The method of claim 9 or 11 wherein said first and second recombination sites share at least 90% sequence identity.

28. The method of claim 9 or 11 wherein said first and second recombination sites share less than 90% sequence identity.

29. The method of claim 9 or 11 wherein said first recombination site comprises a bacterial genomic recombination site and said second recombination site comprises a phage recombination site.

30. The method of claim 29 wherein said bacterial genomic recombination site is attB and said phage recombination site is attP.

31. The method of claim 29, wherein said first recombination site comprises an attB site, and said second recombination site comprises a pseudo-attP site.

32. The method of claim 29, wherein said first recombination site comprises a pseudo-attB site, and said second recombination site comprises an attP site.

33. The method of claim 9 or 11, wherein said recombinase-mediated recombination results in a site that is no longer a substrate for the recombinase.

34. The method of claim 9 or 11 wherein said DNA construct(s) is/are circular.

35. The method of claim 9 or 11 wherein said DNA construct(s) is/are linear.

36. The method of claim 9 wherein said animal is non-human mammal.

37. The method of claim 36 wherein said non-human mammal is a rabbit.

38. The method of claim 9 wherein said animal is a human.

39. A method for producing antibodies in an animal, comprising:
(i) administering at least one DNA expression cassette encoding an antigen, a recombination site, and a recombinase that mediates the integration of said DNA expression cassette into the genome of said animal;
(ii) optionally administering at least one adjuvant;
(iii) harvesting a serum sample after several days from said animal;
(iv) identifying and optionally, purifying antibodies to said administered antigen(s) from said serum sample.

40. The method of claim 39 wherein said adjuvant is introduced as a polypeptide.

41. The method of claim 39 wherein said adjuvant is introduced as a nucleic acid comprising an expression cassette encoding said adjuvant, a recombination site and a recombinase that mediates the integration of said adjuvant encoding-DNA expression cassette into the genome of said animal.

42. The method of claim 39 wherein said animal is a human.

43. The method of claim 39 wherein said animal is a non-human mammal.

44. The method of claim 39 wherein said animal is a non-human transgenic animal carrying an exogenous immunoglobulin translocus.

45. The method of claim 44 wherein said exogenous immunoglobulin translocus is a human or humanized immunoglobulin heavy and/or light chain sequence.

46. The method of claim 44 wherein said non-human transgenic animal is a gene converting animal.

47. The method of claim 44 wherein said non-human transgenic animal is selected from the group consisting of rodents, rabbits, birds including chickens, turkeys, ducks and geese.

48. The method of claim 43 wherein said non-human mammal is a rabbit.

49. The method of claim 39 wherein said antigen is selected from the group consisting of viral, bacterial, fungal, protozoal antigens and antigens associated with diseases like cancer, asthma/allergy, autoimmune diseases, multiple sclerosis, inflammation/sepsis/toxic shock, rheumatoid arthritis, allograft rejections, psoriasis, etc.

50. The method of claim 49 wherein said antigen is CD20.

51. The method of claim 39 wherein said adjuvant is selected from the group consisting of GMCSF, Flt3L, interleukins like IL-1α and β, IL-2, IL-12, IL-15, IL-18, IL-4, IL-5, IL-6, IL-10, TNF-α, TNF-β, IFN-γ, etc. and co-stimulatory molecules like TCA3, CD80 (B7.1), CD86 (B7.2), CD40 ligand (CD154), MCP-1, MIP-1α, β, RANTES, etc.

52. The method of claim 39 wherein said adjuvant is a rabbit GMCSF of SEQ ID NO: 1.
